(19) United States
(12) Patent Application Publication
(10) Pub. No.: US 2004/0127924 A1
(43) Pub. Date: Jul. 1, 2004

(54) Coded microkeratome cutting blade assembly

(76) Inventor: John J. Weidenbenner, Manchester, MO (US)

Correspondence Address: Michael L. Smith, Bausch & Lomb Incorporated, One Bausch & Lomb Place, Rochester, NY, 14604-2701, US

(21) Appl. No.: 10/335,024

(22) Filed: Dec. 30, 2002

(51) Int. Cl. A61F 9/00

(52) U.S. Cl. 606/166

(57) ABSTRACT

A microkeratome blade assembly 10 includes a cutting blade 12 attached to a blade holder 14. Either the blade 12 or blade holder 14 includes indicia representative of a blade extension.

### BACKGROUND OF THE INVENTION

1. Field of the Invention

The present invention relates to cutting blade assemblies and specifically, cutting blade assemblies for use in a microkeratome for use in ophthalmic surgery.

2. Description of the Related Art

Laser-Assisted In-situ Keratomileusis or LASIK surgery has become a widespread and effective eye correction surgical procedure in the last several years. Before a laser ablates a portion of a patient's corneal tissue to correct that patient's vision, a flap of the patient's cornea must be formed.

A typical cornea, on average, is about 520 microns thick. A typical flap thickness for the corneal flap, that is formed prior to laser ablation and LASIK surgery, is desired to be on the order of 160 to 200 microns. As is well known, these corneal flaps are made using microkeratomes that travel in a linear, arcuate, or even in a horizontally hinged path. A microkeratome typically cuts the corneal flap using a cutting blade assembly made with standard razor blade stock available from any of numerous razor blade manufacturers. It is also typical that the cutting blade is oscillated to aid in the cutting, while the cutting blade is translated across the cornea to form a corneal flap.

A rather accurate measurement of the corneal thickness prior to LASIK surgery is obtainable through any number of known measurement methods, such as the use of an ORBSCAN™ Topography System available from Bausch & Lomb Incorporated. After the corneal thickness measurement has been obtained, depending on the surgeon's preference and the amount of correction needed, a flap thickness determination is then chosen by the surgeon.

Typically, in the prior art, each microkeratome comes with a variety of cutting heads, which are precisely manufactured to obtain different flap thicknesses, such as 160 microns, 180 microns, and 200 micron cuts. Again, in the prior art, a single cutting blade assembly has been used with these different precision cutting heads to obtain the different flap thicknesses.

One variation to this is from Med-Logics, Inc.. Med-Logics currently manufactures LASIK blades, which consist of a plano or nominal length blade and a plus and a minus blade, wherein the blade extensions vary from the plano extension either plus or minus 20 microns. According to Med-Logics, this then allows the doctor to produce a flap of thinner or thicker thickness from the plano blade using a given cutting head.

A problem with all prior art microkeratome cutting blade assemblies has been the consistency of the blade extension of the cutting head of the cutting blade assembly. The blade extension is defined as the distance from the cutting tip of the blade to the nearest point of the blade holder. A microkeratome cutting head is precisely machined to applanate the cornea a given amount and to hold the blade holder within fairly tight tolerances. However to this point, the blade extension has not been held to a tight enough tolerance to give a consistent flap thickness cut. The criticality of the blade extension consistency has only recently become understood. However, it has always been a goal to provide a consistent and predictable flap thickness with a given cutting blade in a given microkeratome cutting head.

The consistency of the flap thickness cut is crucial for several reasons. The reasons include that the laser ablation algorithm is based on the patient's need for correction and the amount of stromal bed left to be ablated after the flap has been created. This is critical to achieving an acceptable outcome for the patient. If too much corneal bed is ablated and not enough corneal bed thickness is left, the patient's intraocular pressure could cause serious change to the cornea. Conversely, if the corneal flap is too thin the flap could easily tear or it could be difficult to adequately correct the patient's vision without complications such as halos.

While it is easy to obtain a corneal thickness measurement before LASIK surgery, it has proven extremely difficult to measure corneal thickness of an eye with a corneal flap laid back over, and it is equally difficult to obtain a reliable corneal flap thickness measurement due to changes in hydration of the corneal flap and the cornea which occur quite rapidly under the surgical lights of an operating room.

If the corneal flap is thinner or thicker then desired by the surgeon and a patient's cornea is on the thin side to begin with, then serious complications could result from a flap that is thicker than desired. Therefore, it is desirable to provide a microkeratome cutting blade assembly having a tightly controlled blade extension and to provide an easily accomplished method of producing such a tight blade extension. It is also desirable to provide microkeratome cutting blade assemblies with blade extensions that are coordinated with the cutting head to provide the most consistent, stable, and accurate flap cuts while minimizing any damage to the flap, such as epithelial damage.

Typically, razor blade stock used to form the cutting blade of the cutting blade assembly has a different bevel on either side of the cutting blade. This could potentially affect the consistency of the flap cut, and therefore, it would be desirable to have the cutting blade keyed so that during manufacturing the cutting blade with a given bevel may be formed from one cutting blade assembly to the next. Such keying would also aid in precisely locating the blade relative to the blade holder during manufacturing.

It would also be desirable to provide the cutting blade assemblies with identifying indicia on the cutting blade assembly itself, so that a user may determine the type of cutting blade assembly and its blade extension from the blade assembly itself, rather packaging.

### BRIEF DESCRIPTION OF THE DRAWINGS

FIG. 1 is a side view of a cutting blade assembly in accordance with the present invention;

FIG. 2 is a bottom view of FIG. 1;

FIG. 3 is a bottom view of an alternate embodiment of a cutting blade assembly in accordance with the present invention;

FIG. 4 is a perspective view of yet another alternate embodiment of a cutting blade assembly in accordance with the present invention;

FIG. 5 is a bottom view of yet another alternate embodiment of a cutting blade assembly in accordance with the present invention;

FIG. 6 is a perspective view of a portion of a blade assembly fixture in accordance with the present invention;

FIG. 7 is a cut away partial view of the fixture of FIG. 6;

FIG. 8 is a partial cut away perspective view of the fixture of FIG. 6;

FIG. 9 is a perspective view of an alternate embodiment of a fixture for manufacturing cutting blade assemblies in accordance with the present invention; and

FIG. 10 is a schematic view showing the critical tolerances of a blade extension relative to a cutting head in accordance with the present invention.

### DETAILED DESCRIPTION OF THE DRAWINGS

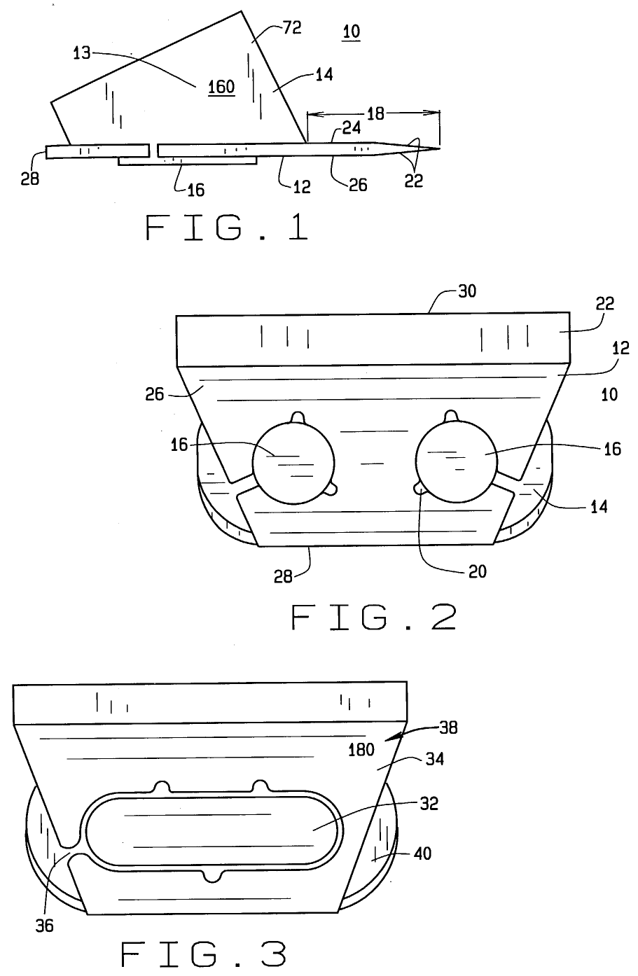

FIG. 1 shows a microkeratome cutting blade assembly 10 in accordance with the present invention. Assembly 10 includes a cutting blade 12 and a blade holder 14 attached to the cutting blade 12. Preferably, blade holder 14 is attached to cutting blade 12 through an aperture or through-hole in cutting blade 12 (not shown) via post member 16 through a commonly known procedure such as heat staking. However, other means of attachment, such as cold staking, the use of adhesives, or other means are also possible. In addition, the aperture does not need to be a through-hole but rather could be mating indentations and raised portions in the blade holder and blade, as is known. The difference between assembly 10 and prior art assemblies, is that a blade extension represented by number 18 is controlled to within at least six (6) ten-thousandths of an inch of a target extension length for assisting and providing a consistent, predictable corneal-flap thickness. Such tight tolerances and blade extensions have been unknown heretofore. For instance, prior art blade assemblies for the Hansatome™ Microkeratome, available from Bausch & Lomb Incorporated, have had blade extensions that varied four (4) thousandths of an inch. It is also preferable that some indicia, such as shown at 13, be placed on the assembly 10. The indicia may be a number, such as the “160” shown at 13 to indicate a depth of cut or flap thickness. The indicia could also include a color coding scheme, where the blade or blade holder is a particular color for a particular blade extension. The blade or blade holder could also contain other makings or indicia to distinguish between blade assemblies with different blade extensions. This use of identifying indicia can be particularly helpful to a surgeon to help ensure that the correct flap cut is made, because the only difference between blade assemblies will be the blade extension 18 length which will be difficult for the naked eye to discern.

FIG. 2 is a bottom view of the assembly 10 of FIG. 1. The blade 12 is placed over post 16 of holder 14 as shown. The view of FIG. 2 is prior to the heat staking, and in this way notches 20 are easily seen. The purpose of notches 20 are to allow the material of post 16 upon heat staking to flow into the notches 20 and therefore, ensure a solid attachment of the blade 12 to the blade holder 14. Preferably, blade holder 14 is made of Lubiloy™ and is molded or machined. Lubiloy™ is a polycarbonate material, which is preferred for blade holder 14, though any known suitable material is acceptable for blade holder 14, such as Delrin™. As previously discussed, cutting blade 12 is preferably formed from razor blade stock widely available from a number of manufacturers. It is noted that there are a different number of notches 20 in the right-hand and left-hand apertures for receiving post 16. The purpose for a different number of notches is to key the cutting blade to aid in ensuring that the forward cutting edge 22 is oriented relative to the blade holder 14 consistently during manufacturing.

By keying the blade 12, it makes it easy for a worker during manufacture to orient the blade in the proper direction so that the bevel of cutting edge 22 is oriented consistently. This may be of particular importance because cutting edge 22 may have different bevels on the topside 24 (see FIG. 1) than on the bottom side 26 of cutting blade 12. This difference in bevels of the forward cutting edge 22 may potentially cause a difference in flap thickness if the orientation of cutting blade 12 were to be flipped. As will be discussed in detail below, it is important that back datum surface 28 be straight and parallel to cutting tip 30.

The keying of the blade assembly may be accomplished in many ways. An alternate embodiment of a cutting blade in accordance with the present invention is shown in FIG. 3 with one oblong post member 32 to attach to a blade 34. The blade 34 may be keyed in at least two manners. The first key is that EDM (Electro-deposition machining) slot 36 is formed on only one (1) side of blade 34. Alternatively, a number or letter insignia such as shown at 38 may be imprinted on one surface of blade 34, in this way blade 34 may be attached to blade holder 40 in a consistent manner to ensure that the bevel angles are always oriented consistently.

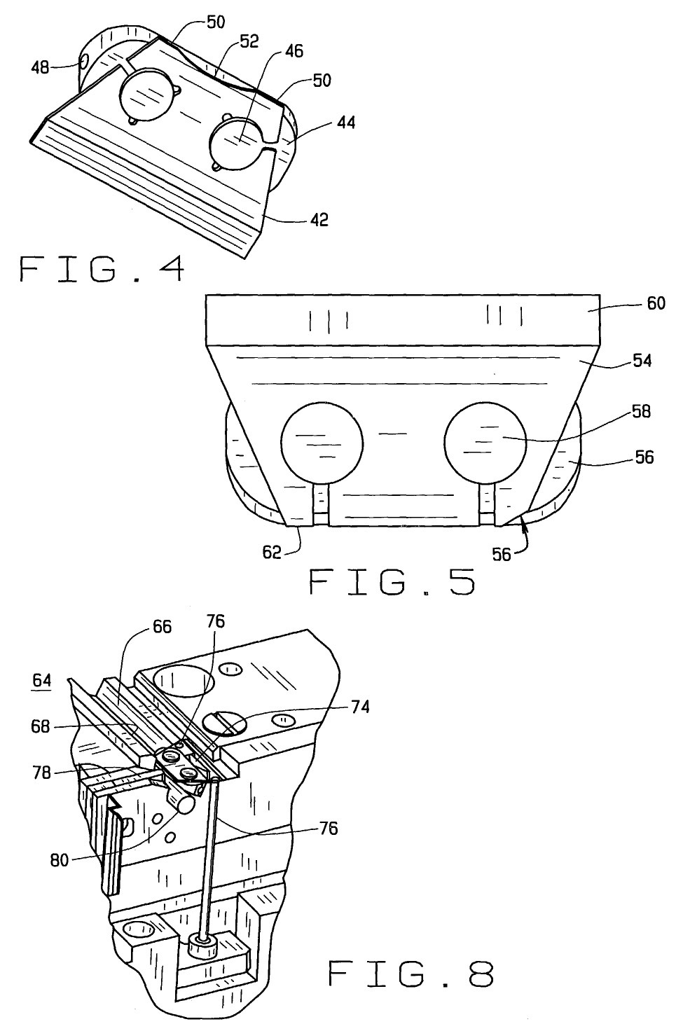

FIG. 4 shows yet another alternative embodiment of a microkeratome cutting blade assembly in accordance with the present invention. A blade 42 is connected to a blade holder 44 via post 46 preferably by heat staking as described above. FIG. 4 also shows an insertion tool hole 48, such as known in the prior art and described in U.S. Pat. No. 6,051,009 to Hellenkamp, et al. Blade 42 has a back datum surface 50 and blade 42 is keyed by radius 52 being offset along back surface 50.

FIG. 5 shows yet another alternative of an embodiment of a microkeratome cutting blade assembly in accordance with the present invention. A blade 54 is attached to a blade holder 56 via post 58, and blade 54 is keyed via the notch 56 formed on one side of the blade 54. Obviously, notch 56 could be formed on blade 54 at any location not along cutting surface 60, that would make blade 54 asymmetric. The cutting blades of FIG. 1 and FIG. 3 are preferred because the cutting blades of FIG. 4 and FIG. 5 would require additional prep work to ensure that no burring or other deformities occur along the back datum edges 50 and 62. It is preferred that at least one aperture is formed in blade 12, such that no part of the aperture is formed in the back datum surface 28. The back datum edges 50 and 62 would require additional work as compared to assemblies shown in FIG. 1 and FIG. 3, because the EDM slots or the radius 52 formed in back datum surface 50 could potentially cause burring or other deformities, and it is essential that the back datum surface be straight during the manufacturing process as will be made clear below. Another reason for keying the blade is that in certain applications it may be desirable to have a fixture that mates with the staking fixture of the blade, such that the blade is always in a known location during manufacturing.

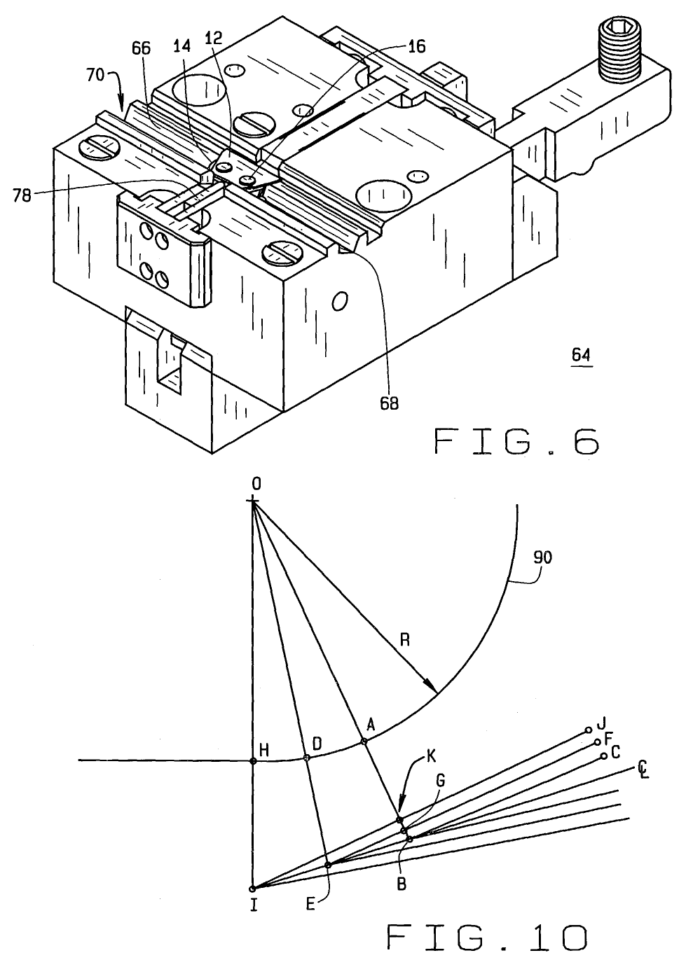

FIGS. 6-8 show the use of a preferred fixture 64 for use in manufacturing microkeratome cutting blade assembly 10 of the present invention. Fixture 64 preferably includes a front datum surface 66 and a back datum surface 68. Initially blade holder 14 is placed within slot 70, such that post 16 is facing upward, as shown. Next, the worker places cutting blade 12 over post 16, as shown. As will be described in further detail, the object of fixture 64 is to hold back datum surface 28 against fixture datum 68, while surface 72 of blade holder 14 is held snugly against front datum surface 66 of fixture 64. Therefore, it is important that back datum surface 28 be free from any burring or other blemishes to ensure that blade 12 is flush against datum surface 68. While the blade 12 and blade holder 14 are held in this position, heat staking is performed on post 16 to attach blade holder 14 to blade 12 in an operation not shown. In this way, blade tolerances in a blade assembly 10 heretofore unknown are accomplished. It is believed that blade extension 18 tolerances can be held to within six (6) ten-thousandths of an inch. This is compared to the prior art tolerances of four (4) thousandths of an inch.

Blade holder 14 and cutting blade 12 are inserted into fixturing device 64 such that cutting blade 12 is adjacent to blade holder 14, wherein mating surfaces on each of the blade holder 14 and blade 12 abut each other. Actuator 74 is caused to move towards back datum surface 68, this pushes the blade holder and via the post 16, the cutting blade 12 and back datum surface 28 is caused to be snugly held against fixture datum surface 68, then clamp posts 76 grasp cutting blade 12 so that cutting blade datum surface 28 abuts fixture datum surface 68 of fixturing device 64 so that the cutting blade is in a known location.

Actuator 74 is then released and actuator 78 and roller 80 are caused to move, such that blade holder 14 moves until it abuts snugly against cutting blade top surface 24 and surface 72 snugly fits against fixture datum surface 66. As can be seen in FIG. 7, actuator 78 pivots about pivot point 82. While blade holder 14 is held in this position against datum surface 66 and cutting blade surface 24, preferably heat staking is performed on post 16 to permanently attach blade holder 14 to cutting blade 12 to form microkeratome cutting blade assembly 10. This heat staking operation is not shown, but is done in a conventional manner.

It is preferred that a number of cutting blade assembly fixtures 64 are attached to a carousel-type device (not shown) so that various operations may be going on at difference points along the carousel while a worker is loading blade holders 14 and cutting blades 12, and removing finished cutting blade assemblies 10 from fixtures 64.

To prevent inopportune loading of a blade holder 14 and cutting blade 12 into the fixture 64, it is preferred that actuator 74 is biased to lie within slot 70 to prevent blade holder 14 from being inserted into fixture 64 except for when fixture 64 is otherwise ready to receive a new blade holder 14 into slot 70.

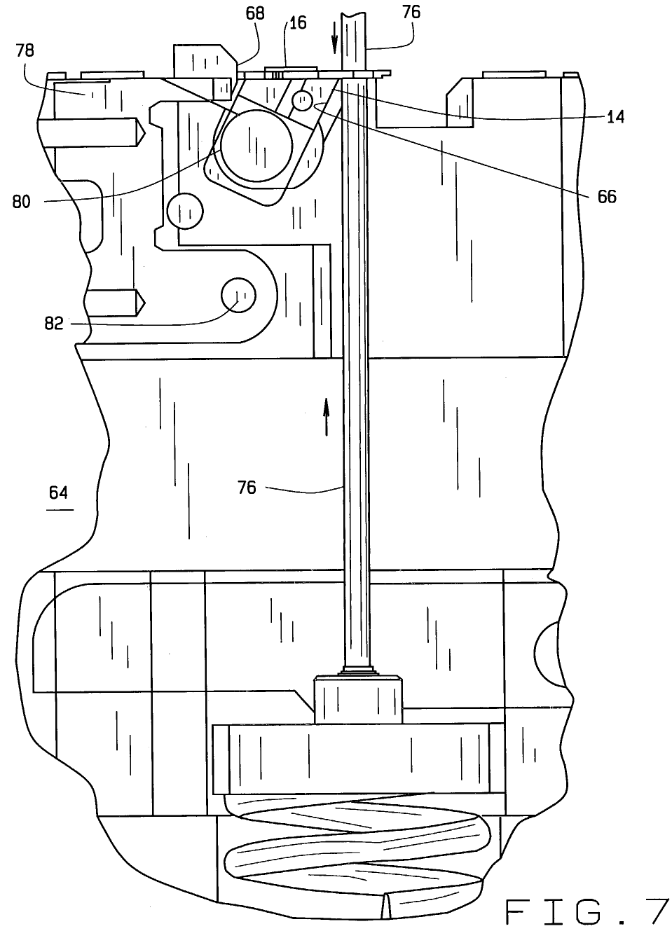

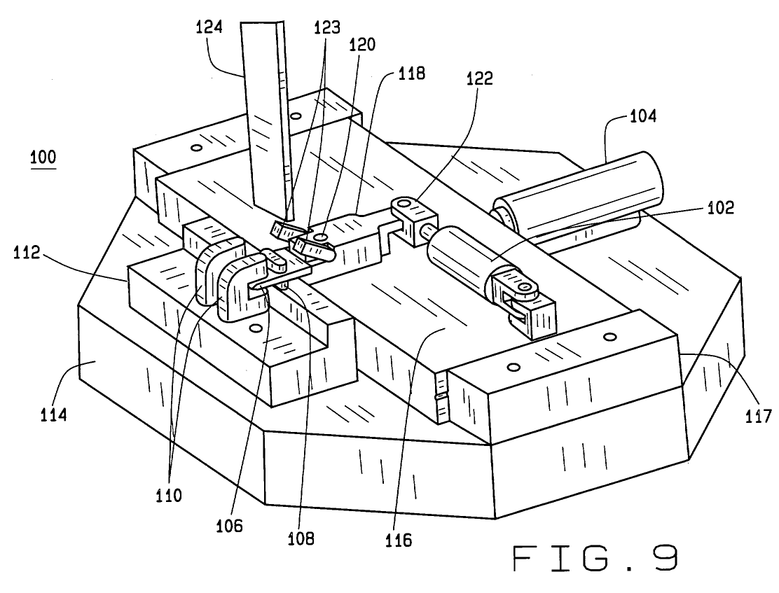

FIG. 9 shows an alternative cutting blade assembly fixture 100 which can be used to make blade assemblies having different blade extension lengths quite easily as compared to the fixture described above at FIGS. 6, 7, and 8. The fixture 64 of FIGS. 6, 7, and 8 in order to produce blade extensions of different lengths, a new datum surface 66 or 68 of different orientation would be required. Whereas instead of the hardware change required of fixture 64 to produce a different blade extension length, a simple software instruction (controls not shown)could be made to fixture 100. Two closed-loop actuators 102 and 104 provide alignment of the blade 106 to the blade holder 108 during an assembly process. Actuator 104 controls the blade extension and actuator 102 controls parallelism of the blade holder to the front cutting edge of blade 106. The location of the blade holder 108 is a known distance from the optical emitters and receivers 110. Emitters/receivers 110 feed information for blade extension and parallelism and must have variable measurement capability to be able to accommodate assembly requirements for different blade extensions. An alternative would be change the location relationship of the blade holder to the emitters/receivers 110 for each desired plate extension, similar to the need for changing datum surfaces 66 and 68 referenced above.

Preferably optical emitters/receivers 110 are attached to mount 112 which is consequently attached to base 114.

Blade holder carriage 116, holds blade parallel actuator 102 and blade parallel mechanism 118 for ensuring that a front edge of the blade holder is parallel to the cutting edge of the blade 106. Upon receiving measurement information, actuator 102 causes mechanism 118 to pivot about pivot point 120 until the distance from the blade holder to the cutting edge is the same in both the emitters/receivers 110. Actuator 102 is operably connected to mechanism 118 via linkage 122. Once the blade holder 108 and blade 106 are parallel, actuator 104 causes blade holder carriage 116 to move until the blade extension measurement from emitters/receivers 110 is at the desired extension length. Preferably blade holder carriage 116 slidably moves within precision guide 117. Once blade 106 is parallel to blade holder 108, gripping mechanism 123 grabs the blade to hold it in place relative to the blade holder 106.

Once the proper blade extension has been achieved then blade staking may be accomplished by tool 124 or by other means such heat staking. It is also possible that, in addition to staking, the blade holder to the blade, tool 124 may be used to coin a blade extension indicia onto the completed blade assembly.

It has come to be appreciated that in relation to a cutting head 90, a portion of which is shown in FIG. 10, there is a precision zone which defines an area in which the most reliable and consistent flap thickness cuts may be achieved with a cutting blade. This precision zone is shown in FIG. 10 as being defined by line O-I on one extreme and O-B on the other extreme. A typical microkeratome cutting head 90 has a radius R which is at the trailing end of an applanation area 92. There are shown three (3) blade positions within the precision zone defined by the first blade position defined by line B-C, the second blade position defined by line E-F, and the third blade position defined by line I-J. The thinnest flap position is at the position defined by blade surface B-C which is also the shortest blade extension position. It has been determined that the most advantageous and reliable and accurate blade cuts will be made at this boundary defined by line O-B, at a point where line O-B is perpendicular to a blade surface B-C. Any blade shorter then this configuration may result in a flap being cut in an unstable region due to the rapid expansion of the cutting distance caused by radius R. As a blade extension is increased, the cut thickness also increases to a maximum point of reliability and accurately, defined by line O-I. As the cut distance increases with blade extension, the flap translates through a smaller section slightly compressing the flap. If the blade extends beyond the tangent point of the radius defined by line O-I, the compression may be too great and there is significant risk of damaging the epithelial layer of a corneal flap. The present invention also consists of a head and blade coordinated design which keeps the blade extension and range of flap thickness between the minimum radial gap defined by line O-B and maximum extension defined by a vertical position to the tangent point O-I.

In this way, a plurality of cutting blade assemblies can be formed such that a variety of blade extension lengths are formed and coordinated to be used with a single microkeratome cutting head 90. A variety of corneal flap thicknesses may be formed with the single microkeratome cutting head 90 when the assemblies are placed in the cutting head 90 and the blade assembly's cutting edge is within a precision zone relative to the cutting head 90 for minimizing corneal flap compression and maximizing a consistency of flap thickness.

I claim:

1. A microkeratome cutting blade assembly comprising:
a cutting blade;
a blade holder attached to the cutting blade; and
wherein one of the blade and blade holder includes indicia representative of a blade extension.

2. The assembly of claim 1, wherein the indicia includes numbers or letters for a given blade extension.

3. The assembly of claim 1, wherein the indicia includes a specific color deposed on the blade assembly for a given blade extension.
